(19) United States
(12) Patent Application Publication
(10) Pub. No.: US 2007/0010525 A1
(43) Pub. Date: Jan. 11, 2007

(54) Method and compositions for modulating neuropeptide hormone secretion

(76) Inventor: Meyer Jackson, Madison, WI (US)

Correspondence Address: BAKER DONELSON BEARMAN CALDWELL & BERKOWITZ, PC, 555 11TH STREET, NW, 6TH FLOOR, WASHINGTON, DC, 20004, US

(21) Appl. No.: 11/475,010

(22) Filed: Jun. 27, 2006

Related U.S. Application Data
(60) Provisional application No. 60/693,939, filed on Jun. 27, 2005.

(51) Int. Cl. A61K 31/519

(52) U.S. Cl. 514/252.16; 514/262.1

(57) ABSTRACT

Methods for increasing the release of oxytocin or vasopressin or both from the posterior pituitary of a mammal in need thereof, comprising administering to the mammal an effective amount of a cyclic guanosine 3′,5′-monophosphate phosphodiesterase type five (cGMP PDE5) inhibitor to the mammal. The method is particularly useful to treat pregnant female or postnatal mammal wherein labor, fetal expulsion, or milk let-down or production needs to be induced, enhanced or augmented. Also provided are pharmaceutical compositions suitable for the inventive method.

### CROSS REFERENCE TO A RELATED APPLICATION

This application claims priority to U.S. Provisional Application No. 60/693,939 filed Jun. 27, 2005, the content of which is incorporated herein by reference in its entirety.

### FEDERAL GOVERNMENT INTEREST

This invention was made with United States government support under a grant from the National Institutes of Health (NIH), Grant Number NIH NS030016. The United States has certain rights to this invention.

### FIELD OF THE INVENTION

The application relates to methods and compositions for modulating the release of neuropeptide hormones, in particular oxytocin and vasopressin, by the posterior pituitary gland.

### BACKGROUND OF THE INVENTION

The pituitary gland is a small structure located at the base of the brain. In humans and other vertebrates, it consists of two lobes: the anterior lobe and the posterior lobe. The anterior lobe contains at least five types of secretory cells. Each of these cells secretes its hormones in response to hormones reaching them from the hypothalamus of the brain.

The posterior lobe of the pituitary releases two closely related hormones, oxytocin (OT) and antidiuretic hormone (ADH), both synthesized in the hypothalamus, stored in the posterior pituitary, and released into the circulation as needed.

OT is a short-lived, fast acting neuropeptide of 9 amino acids. Its function includes stimulation of smooth muscle contraction during child birth (Soloff, “Endocrine control of parturition and lactation,” In: Wynn R M, Jollie W P (eds) Biology of the Uterus, 2nd ed., Plenum Press, NY pp 559-607, 1989; Fuchs et al., “Oxytocin receptors and human parturition. A dual role for oxytocin in the initiation of labor,” Science, 215:1396-1398, 1982), stimulation of constriction of blood vessels and enhancement of sensitivity of some tissues to other hormones and nerves. The main tissues affected by OT are the uterus, including endometrium and myometrium, vagina, breasts (both sexes), erectile tissue (both sexes), and seminal vesicles. It also enhances uterine muscle contractions in orgasm, the vascular constriction that lessens placental separation bleeding, the let-down reflex that nursing mothers have when babies cry or suckle (Soloff et al., “Oxytocin receptors: Triggers for parturition and lactation,” Science, 204:1313-1315, 1979), and prostaglandin release from endometrium/decidua and the amnion (Hinko and Soloff, “Up-regulation of oxytocin receptors in rabbit amnion by glucocorticoids: Potentiation by cyclic adenosine 3′,5′-monophosphate,” Endocrinology, 133:1511-1519, 1993).

On the other hand, the release of oxytocin from the posterior pituitary is also known to be stimulated by sensory stimuli arising from the cervix, vagina, and breast. Secretion of oxytocin is also stimulated by increases in the osmolality of plasma, but is suppressed by ethanol and ovarian relaxin.

Oxytocin is currently indicated for stimulation of uterine contraction to induce labor and for the control of postpartum hemorrhage following delivery of the placenta. It is also indicated for stimulation of lactation for breast-feeding. Oxytocin is currently prepared synthetically and sold under various trade names including Pitocin™ (Parke-Davis, Morris Plains, N.J.) and Syntocinon™. It can be administered intravenously, intramuscularly, and by nasal absorption. Activity of oxytocin is expressed in terms of USP units, as defined in a bioassay of uterine-stimulating potency of posterior pituitary extracts. One USP unit is the equivalent of approximately 2 μg of pure peptide.

As oxytocin can produce contractions in the collecting ducts of the mammary glands with the resulting ejection of milk, it is also widely used in effecting or increasing milk production in farm animals.

Relatedly, it has been suggested to be used to achieve controlled parturition in farm animals such as cattle, which has long been a subject of veterinary investigation. Parturition (expulsion of the fetus from the uterus) requires both contraction of the myometrium, the smooth muscle of the uterus, and a softening of the connective tissue of the cervix, so that it will stretch and dilate sufficiently (a process known as “ripening”), to allow the fetus to be expelled. Aside from occasional clinical reasons for inducing labor before its spontaneous onset, in large dairy herds, a controlled parturition regimen is economically desirable as births could be planned and managed.

In vivo, however, the biochemical half-life of injected oligopeptide hormones such as oxytocin is only a few minutes and the duration of the myometrial response to single injections is only slightly longer. While the duration of uterine response can be prolonged by giving large doses of oxytocin, attempts to prolong the effect by giving excessive doses can result in uterine tetany or tachyphylaxis, both of which, as with prostaglandins, endanger the mother and fetus. Ideally the most physiological approach to prolonging oxytocin action until the required clinical task is accomplished would be to administer it by continuous i.v. infusion. This is practiced in human obstetrics. In human use, OT is given in continuous infusion at 1 ml/min of a solution of 20 IU/l.—which represents a rate of about 40 ng or pmol/min—in 5% glucose, but constant monitoring of uterine contractions and fetal heartbeat are required to prevent tetany and fetal damage.

Long term infusion in farm animals, however, is hardly possible under ordinary conditions of veterinary medical practice with large animals. Reports of use in bovine obstetrics vary from a failure to induce labor with i.v. or i.m. injection of 100 IU (=about 0.2 mg.) to successful induction with i.v. infusion of only 4-5 IU over 1 hour (but in only 3 animals). Aside from the inconsistent results reported in the literature and the need for continuous monitoring when OT is administered via infusion, the procedure is practically impossible if routine long term infusions in large animals is required.

Thus, there is a need for methods and compositions that would achieve increased OT level in mammals, especially for inducing or enhancing labor, and increasing milk production on a long term basis. Preferably, such method will avoid the above-discussed shortcomings of long term administration of i.v. injection.

In addition, because OT is naturally released by the posterior pituitary in pulses of about 5 minutes apart, continuous i.v. injection of OT is harsh on pregnant women. As discussed above, labor is induced when there is maternal illness, fetal distress, or, more commonly, prolonged pregnancy. Oxytocin is currently administered to induce active labor, to stimulate weak contractions in labor, and to cause contraction of the uterus after delivery of the placenta. Induction of labor can result in uterine hyperstimulation, and poor neonatal outcome. Oxytocin administration causes a steady sustained increase in the circulating level of the hormone. During normal parturition secretion of oxytocin by the posterior pituitary is pulsatile; release occurs at well separated intervals varying from five to ten or more minutes. Elaborate efforts have been made to mimic this process by administering oxytocin in larger doses at well-timed intervals, yet high dose bolus administration remains difficult and risky.

Thus, there is a further need for methods and compositions to increase release of OT from the posterior pituitary in a more natural manner. Specifically, there is a need for a method and a pharmaceutical composition that will enhance oxytocin release by increasing the release elicited by the naturally occurring pulsatile electrical activity in the posterior pituitary.

ADH, also known as [Arg⁸]-vasopressin (AVP), arginine vasopressin or the neurohypophyseal peptide, is also a peptide of 9 amino acids with sequence homology with oxytocin. ADH is involved in diverse functions, including the contraction of smooth muscle, stimulation of liver glycogenolysis, modulation of corticotropin release from the pituitary, and inhibition of diuresis (Michellet al., 1979, Hormonal stimulation of phosphatidylinositol breakdown, with particular reference to the hepatic effect of vasopressin. Biochem. Soc. Trans. 7:861-865).

These physiological effects are mediated through the binding of AVP to specific membrane receptors of the target cells. ADH receptors are G protein-coupled and have been divided into at least three types: V1a, V1b, and V2. The V1a (vascular/hepatic) and V1b (anterior pituitary) receptors act through phosphatidylinositol hydrolysis to mobilize intracellular Ca²⁺ (Jard, S. et al. 1986. Vasopressin antagonists allowed the demonstration of a novel type of vasopressin receptor in the rat adenohypophysis. Mol. Pharmacol. 30:171-177). The V1a receptor mediates physiological effects such as cell contraction and proliferation, platelet aggregation, coagulation factor release, and glycogenolysis. The V1b receptor exists in the anterior pituitary, where it stimulates corticotropin release. The V2 receptors are found primarily in the kidney. They are linked to adenylate cyclase and the production of cAMP, and are associated with antidiuresis (Thibonnier, M. 1988. Vasopressin and blood pressure. Kidney Int. 34:S52-S56). All of these receptors have been cloned (More et al, 1992. Molecular cloning and expression of a rat V1a arginine vasopressin receptor. Nature. 356:523-526; Lolait et al., 1992. Cloning and characterization of a vasopressin V2 receptor and possible link to nephrogenic diabetes insipidus. Nature. 357:336-339; Keyzer et al., 1994, Cloning and characterization of the human V3 pituitary vasopressin receptor. FEBS Lett. 356:215-220) and belong to the family of “seven membrane-spanning” receptors, which signal through G proteins (Thibonnier et al., 2002, Molecular pharmacology and modeling of vasopressin receptors. Prog. Brain Res. 139:179-196).

ADH is synthesized primarily in the magnocellular neurons of the hypothalamic paraventricular nuclei and in the supraoptic nuclei that project to the posterior pituitary. In addition, parvocellular neurons of the paraventricular nuclei coexpressing ADH and corticotropin-releasing hormone (CRH) coordinate hypothalamic-pituitary-adrenal (HPA) system activity and project to the external layer of the median eminence, where AVP and CRH are released into the portal blood (Antoni, 1993, Vasopressinergic control of pituitary adrenocorticotropin secretion comes of age. Front. Neuroendocrinol. 14:76-122). Numerous investigations have shown that ADH synergizes potently with CRH to stimulate pituitary adrenocorticotropic hormone (ACTH) release both in vitro and in vivo (see e.g. Antoni, 1993, supra). A recent study using mice lacking the type 1 CRH receptor gene (Crhr1−/− mice) further provided indirect evidence that this vasopressinergic system can work as a compensatory mechanism to maintain HPA activity when CRH/CRHR1 signaling is impaired (Turnbull et al., 1999, CRF type 1 receptor-deficient mice exhibit a pronounced pituitary-adrenal response to local inflammation. Endocrinology. 140:1013-1017; Muller et al., 2000, Selective activation of the hypothalamic vasopressinergic system in mice deficient for the corticotropin-releasing hormone receptor 1 is dependent on glucocorticoids. Endocrinology. 141:4262-4269). Thus, ADH appears to regulate HPA axis activity in modulating the effect of CRH; however, its role is not fully understood. Tanoue et al., (2004, The vasopressin V1b receptor critically regulates hypothalamic-pituitary-adrenal axis activity under both stress and resting conditions. J. Clin. Invest. 113:302-309) demonstrated that the V1b receptor plays a crucial role in regulating hypothalamic-pituitary-adrenal axis activity by maintaining ACTH and corticosterone levels, not only under stress but also under basal conditions.

ADH acts on the collecting ducts of the kidney to facilitate the reabsorption of water into the blood. This reduces the volume of urine formed (giving it its name of antidiuretic hormone). A deficiency of ADH or inheritance of mutant genes for its receptor (called V2) leads to excessive loss of urine, a condition known as diabetes insipidus. The most severely-afflicted patients may urinate as much as 30 liters of urine each day. The disease is accompanied by terrible thirst, and patients must continually drink water to avoid dangerous dehydration.

Thus, there is a need for methods and compositions for the treatment of ADH deficiency. Such methods and compositions can be used to treat diseases such as diabetes insipidus, reversing hypotension or hemorrhage, treating urinary incontinence or bedwetting. Preferably, the methods and compositions should mimic the effects of natural vasopressin release by the posterior pituitary.

There is also a need for methods and compositions that can be used to stimulate the release of oxytocin or vasopressin or both from the posterior pituitary.

### SUMMARY OF THE INVENTION

In one embodiment, the present invention provides methods and compositions that increase oxytocin release from the posterior pituitary while maintaining the natural pulsatility, and provides a more natural method of inducing labor, enhancing contractions, and inducing uterine contractions after delivery of the placenta. Specifically, the present invention provides a method for increasing the release of oxytocin or vasopressin from the posterior pituitary of a mammal in need thereof, the method comprising administering to the mammal an effective amount of a cyclic guanosine 3′,5′-monophosphate phosphodiesterase type five (cGMP PDE5) inhibitor.

In one embodiment, the mammal is a pregnant female mammal and labor, fetal expulsion, or milk let-down is induced, enhanced or augmented in the mammal. For example, the female mammal is a near-term, full-term or over-term pregnant woman and wherein labor is induced or enhanced. Alternatively, is a farm animal such as a pig or a cattle. In another embodiment, the method of the present invention to induce, enhance or augment milk-let down in a prenatal, neonatal or postnatal female mammal, such as a breast-feeding woman would or a mild-producing dairy cow, goat, or sheep.

A suitable PDE5 inhibitor for the method of the present invention may be selected from the group consisting of: pyrazolo (4,3-d)pyrimidin-7-ones; isomeric pyrazolo (3,4-d)pyrimidin-4-ones; quinazolin-4-ones; pyrido (3,2-d)pyrimidin-4-ones; purin-6-ones; and pyrazolo (4,3-d)pyrimidin-4-ones, especially 3-ethyl-5-(5-(4-ethylpiperazin-1-ylsulphonyl)-2-n-propoxyphenyl)-2-(pyridin-2-yl)methyl-2,6-dihydro-7H-pyrazolo(4,3-d)pyrimidin-7-one (sildenafil), (2-[2-ethoxy-5-(4-ethylpiperazine-1-sulfonyl)-phenyl]-5-methyl-7-propyl-3H-imidazo [5,1-f][1,2,4]triazin-4-one) (vardenafil), or Pyrazino[1′,2′:1,6]pyrido[3,4-b]indole-1,4-dione,6-(1,3-benzodioxol-5-yl)-2,3,6,7,12,12a-hexahydro-2-methyl-,(6R, 12aR)-6R-trans)-6-(1,3-benzodioxol-5-yl)-2,3,6,7,12,12a-hexahydro-2-methyl-pyrazino[1′,2′:1,6]pyrido[3,4-b]indole-1,4-dione (tadalafil).

It is to be understood that the compounds listed above may be in the form of their racemates, their pure stereoisomers, in particular enantiomers or diastereomers, or in the form of mixtures of stereoisomers, in particular the enantiomers or diastereomers, in any mixing ratio; in the illustrated form or in the form of suitable acids or bases or salts, in particular physiologically acceptable salts, or in the form of suitable solvates, in particular the hydrates.

In another embodiment, the present invention provides a pharmaceutical composition suitable for a treatment method that is in need of increasing the release of oxytocin or vasopressin from the posterior pituitary of a mammal. The pharmaceutical composition of the present invention comprises an effective amount of a cyclic guanosine 3′,5′-monophosphate phosphodiesterase type five (cGMP PDE5) inhibitor, as described and exemplified above, and a pharmaceutically acceptable excipient. Preferably, the composition is specifically formulated and dosed for the treating female mammals that are pregnant and in need of induction, enhancement or augmentation of labor or fetal expulsion. In a further embodiment, the composition of the present invention is specifically formulated and dosed for treating prenatal, neonatal or postnatal female mammals where milk let-down desired to be induced, enhanced or augmented.

### BRIEF DESCRIPTION OF THE FIGURES

FIG. 1 shows that sildenafil produces a large and significant increase in evoked oxytocin release.

FIG. 2 shows the enhancement by sildenafil of the effect of cGMP on potassium current resulting in the enhancement of oxytocin release.

### DESCRIPTION OF THE INVENTION

The NO/cGMP signaling cascade plays important roles in regulating a wide range of cellular functions including muscle relaxation, cardiovascular function, intestinal secretion, neurite outgrowth, and synaptic transmission. The elucidation of this molecular signaling pathway was a major advance in molecular endocrinology, as evidenced by the award of the Nobel Prize in Physiology and Medicine in 1998 to Robert Furchgott, Louis Ignarro, and Ferid Murad. The basic signal transduction process is initiated by an elevation in cytosolic calcium. Calcium then activates the enzyme nitric oxide synthase. This enzyme produces nitric oxide (NO). Newly created NO then binds to the enzyme guanylate synthase, activating the enzyme to produce cyclic GMP (cGMP). cGMP activates a specific protein kinase, cGMP-dependent protein kinase (PKG), and PKG then phosphorylates a variety of proteins to change their function. For example, phosphorylation of ion channel proteins changes the way they open and close so that the electrical excitability of a cell can be controlled and regulated. Although much is known about the roles of the NO/cGMP in controlling ion channels and electrical excitability (Ahern et al., 2002, cGMP and S-nitrosylation: two routes for modulation of neuronal excitability by NO. Trends in Neuroscience 25:510-517), relatively little is known about how these signaling molecules regulate the release of peptide hormones.

NO degrades rapidly by spontaneous chemical oxidation. By contrast, cGMP degradation is under biological control by a variety of phosphodiesterase (PDE) enzymes. This makes PDEs an important regulator of the strength of the response to activation of the NO/cGMP signaling cascade. There are a number of different types of PDEs, one of which is known as PDE5. Thus, in cells where PDE5 assumes the role of terminating a response of the NO/cGMP cascade, specific PDE5 inhibitors such as sildenafil will enhance the response.

It has been now surprisingly discovered that PDE5 is responsible for regulating NO/cGMP cascade in the posterior pituitary and that PDE5 inhibitor can be used to achieve enhancement of these responses to the NO/cGMP cascade. Specifically, PDE5 inhibitors have been surprisingly discovered to enhance the release of oxytocin from the posterior pituitary.

Accordingly, in one embodiment, the current invention provides methods and pharmaceutical compositions which can be used to more naturally control, manipulate, induce or enhance/augment labor, including inducing labor in late pregnancies in a natural, safe and reproducible way. The methods and compositions of the present invention enable control over the labor progression which up to now has not been unavailable. The method of the present invention comprises administering to a pregnant woman a PDE5 inhibitor, alone or in a suitable combination with other agents and with pharmaceutically acceptable excipients.

The method of the present invention can be used for induction of labor at term (time of ordinary birth), enhancement or augmentation of labor (thereby speeding up fetal expulsion and child delivery), induction of labor in connection with a pathological pregnancy (e.g. fetal malformation, intrauterine fetal death), induction of labor for other medical reasons, management of prolonged labor due to cervical dystocia, induction of cervical ripening of a non-pregnant female or pregnant female to assist for surgical or diagnostic procedure, and induction of cervical ripening for female to be treated by in vitro fertilization.

It is known that oxytocin and vasopressin play a role in learning and memory. For example, Croiset et al., 2000, (European Journal of Pharmacology 405:225-234), provides a review of reports showing that exogenous vasopressin enhances sympathetic nervous system activity. Tomizawa et al., 2003 (Nature Neuroscience 6:384-390) showed that oxytocin improves long lasting spatial memory during motherhood through the MAP kinase cascade. Accordingly, the present inventive method may also be used for enhancing memory.

In yet another embodiment the current invention provides pharmaceutical compositions comprising an effective amount of at least a PDE5 inhibitor, together with a physiologically- and/or pharmaceutically-acceptable carrier, excipient, or diluent, optionally with one or more other suitable pharmaceutically effective ingredients. The compositions are useful for induction of labor in near-term, full-term or over-term pregnancy, induction of labor or to speed up parturition or fetal expulsion. The compositions are administered to a pregnant woman alone or in combination with other pharmaceutically effective agents.

Suitable agents that may be administered together with a PDE5 inhibitor of the present invention may be selected from the group consisting of other anti-gestational agents, anesthetics, and mixtures thereof.

The composition of the present invention may also be used in combination with analgesics, such as acetaminophen, acetylsalicylic acid, morphine, fentanyl, or other similar acting

Other agents used to induce labor include prostaglandins and progesterone antagonists.

The present composition may also include other agents that are typically used for administration to a pregnant patient in need of labor induction and/or augmentation or to counter the side effects of the ingredients present therein. For example, the method of the invention may be used in connection with mechanical methods of inducing or enhancing labor.

In a further embodiment, the present invention provides compositions and methods for increasing milk let down in mammals, which comprises administering to said mammal an effective amount of a PDE5 inhibitor. As discussed above, PDE5 inhibitors increase the release of oxytocin from the posterior pituitary. Oxytocin circulates to the udder to induce the release of milk, enhancing milk output. The time required to initiate the milk letting down reflex will also be shortened, reducing the time necessary for udder stimulation. This shortening of time for milking may be advantageous in reducing mastitis and other complications associated with mechanical milking.

The compositions and methods of the present invention can be used for increasing milk production, and in particular treating agalactia post partum and lactation failure in mammals, including farm animals. Agalactia post partum is characterized by partial or complete lactation failure one to three days after parturition. Other symptoms such as elevated rectal temperature, depression, reduced appetite and mastitis are often observed.

Unlike compositions in the prior art, the compositions of the present invention maintain their activity for a long period after administration or application. The composition of the present invention can be used to increase milk production in a subject in need thereof.

Many PDE5 inhibitors are known in the art and are suitable for the preparation of pharmaceutical compositions of the present invention or for use in the methods of the present invention. Preferably, the PDE5 inhibitors suitable for the present invention is a cGMP specific PDE5 inhibitor (cGMP PDE5 inhibitor). Preferably, they include: the pyrazolo [4,3-d]pyrimidin-7-ones disclosed in EP-A-0463756; the pyrazolo [4,3-d]pyrimidin-7-ones disclosed in EP-A-0526004; the pyrazolo [4,3-]pyrimidin-7-ones disclosed in published international patent application WO 93/06104; the isomeric pyrazolo [3,4-d]pyrimidin-4-ones disclosed in published international patent application WO 93/07149; the quinazolin-4-ones disclosed in published international patent application WO 93/12095; the pyrido [3,2-d]pyrimidin-4-ones disclosed in published international patent application WO 94/05661; the purin-6-ones disclosed in published international patent application WO 94/00453; the pyrazolo [4,3-d]pyrimidin-7-ones disclosed in published international patent application WO 98/49166; the pyrazolo [4,3-d]pyrimidin-7-ones disclosed in published international patent application WO 99/54333; the pyrazolo [4,3-d]pyrimidin-4-ones disclosed in EP-A-0995751; the pyrazolo [4,3-d]pyrimidin-7-ones disclosed in published international patent application WO 00/24745; the pyrazolo [4,3-d]pyrimidin-4-ones disclosed in EP-A-0995750; the compounds disclosed in published international application WO95/19978; the compounds disclosed in published international application WO 99/24433 and the compounds disclosed in published international application WO 93/07124; the pyrazolo [4,3-d]pyrimidin-7-ones disclosed in published international application WO 01/27112; the pyrazolo [4,3-d]pyrimidin-7-ones disclosed in published international application WO 01/27113; the compounds disclosed in EP-A-1092718; and the compounds disclosed in EP-A-1092719.

Other V phosphodiesterase inhibitors for the use according to the present invention include: 5-[2-ethoxy-5-(4-methyl-1-piperazinylsulphonyl) phenyl]-1-methyl-3-n-propyl-1,6-dihydro-7H-pyrazolo [4,3-d]pyrimidin-7-one (sildenafil), also known as 1-[[3-(6,7-dihydro-1-methyl-7-oxo-3-propyl-1H-pyrazolo[4,3-d]pyrimidin-5-yl)-4-ethoxyphenyl]sulphonyl]-4-methylpiperazine (see EP-A-0463756); 5-(2-ethoxy-5-morpholinoacetylphenyl)-1-methyl-3-n-propyl-1,6-dihydro-7H-pyrazolo[4,3-d]pyrimidin-7-one (see EP-A-0526004); 3-ethyl-5-[5-(4-ethylpiperazin-1-ylsulphonyl)-2-n-propoxyphenyl]-2-(pyridin-2-yl)methyl-2,6-dihydro-7H-pyrazolo[4,3-d]pyrimidin-7-one (see WO98/49166); 3-ethyl-5-[5-(4-ethylpiperazin-1-ylsulphonyl)-2-(2-methoxyethoxy)pyridin-3-yl]-2-(pyridin-2-yl)methyl-2,6-dihydro-7H-pyrazolo[4,3-d]pyrimidin-7-one (see WO99/54333); (+)-3-ethyl-5-[5-(4-ethylpiperazin-1-ylsulphonyl)-2-(2-methoxy-1(R)-methylethoxy)pyridin-3-yl]-2-methyl-2,6-dihydro-7H-pyrazolo[4,3-d]pyrimidin-7-one, also known as 3-ethyl-5-{5-[4-ethylpiperazin-1-ylsulphonyl]-2-([(1R)-2-methoxy-1-methylethyl]oxy)pyridin-3-yl}-2-methyl-2,6-dihydro-7H-pyrazolo[4,3-d]pyrimidin-7-one (see WO99/54333); 5-[2-ethoxy-5-(4-ethylpiperazin-1-ylsulphonyl)pyridin-3-yl]-3-ethyl-2-[2-methoxyethyl]-2,6-dihydro-7H-pyrazolo[4,3-d]pyrimidin-7-one, also known as 1-{6-ethoxy-5-[3-ethyl-6,7-dihydro-2-(2-methoxyethyl)-7-oxo-2H-pyrazolo[4,3-d]pyrimidin-5-yl]-3-pyridylsulphonyl}-4-ethylpiperazine (see WO 01/27113, Example 8); 5-[2-iso-Butoxy-5-(4-ethylpiperazin-1-ylsulphonyl)pyridin-3-yl]-3-ethyl-2-(1-methylpiperidin-4-yl)-2,6-dihydro-7H-pyrazolo[4,3-d]pyrimidin-7-one (see WO 01/27113, Example 15); 5-[2-Ethoxy-5-(4-ethylpiperazin-1-ylsulphonyl)pyridin-3-yl]-3-ethyl-2-phenyl-2,6-dihydro-7H-pyrazolo[4,3-d]pyrimidin-7-one (see WO 01/27113, Example 66); 5-(5-Acetyl-2-propoxy-3-pyridinyl)-3-ethyl-2-(1-isopropyl-3-azetidinyl)-2,6-dihydro-7H-pyrazolo[4,3-d]pyrimidin-7-one (see WO 01/27112, Example 124); 5-(5-Acetyl-2-butoxy-3-pyridinyl)-3-ethyl-2-(1-ethyl-3-azetidinyl)-2,6-dihydro-7H-pyrazolo[4,3-d]pyrimidin-7-one (see WO 01/27112, Example 132); (6R, 12aR)-2,3,6,7,12,12a-hexahydro-2-methyl-6-(3,4-methylenedioxyphenyl)-pyrazino[2′,1′:6,1]pyrido[3,4-b]indole-1,4-dione (IC-351), i.e. the compound of examples 78 and 95 of published international application WO95/19978, as well as the compound of examples 1, 3, 7 and 8; 2-[2-ethoxy-5-(4-ethyl-piperazin-1-yl-1l-sulphonyl)-phenyl]-5-methyl-7-propyl-3H-imidazo [5,1-f][1,2,4]triazin-4-one (vardenafil) also known as 1-[[3-(3,4-dihydro-5-methyl-4-oxo-7-propylimidazo [5,1-f]-as-triazin-2-yl)-4-ethoxyphenyl]sulphonyl]-4-ethylpiperazine, i.e. the compound of examples 20, 19, 337 and 336 of published international application WO99/24433; and the compound of example 11 of published international application WO93/07124; and compounds 3 and 14 from Rotella D P, J. Med. Chem., 2000, 43, 1257.

Still other type cGMP PDE5 inhibitors useful in conjunction with the present invention include: 4-bromo-5-(pyridylmethylamino)-6-[3-(4-chlorophenyl)-propoxy]-3(2H)pyridazinone; 1-[4-[(1,3-benzodioxol-5-ylmethyl)amiono]-6-chloro-2-quinozolinyl]-4-piperidine-carboxylic acid, monosodium salt; (+)-cis-5,6a,7,9,9,9a-hexahydro-2-[4-(trifluoromethyl)-phenylmethyl-5-methyl-cyclopent-4,5]imidazo[2,1-b]purin-4(3H)one; furazlocillin; cis-2-hexyl-5-methyl-3,4,5,6a,7,8,9,9a-octahydrocyclopent[4,5]-imidazo[2,1-b]purin-4-one; 3-acetyl-1-(2-chlorobenzyl)-2-propylindole-6-carboxylate; 3-acetyl-1-(2-chlorobenzyl)-2-propylindole-6-carboxylate; 4-bromo-5-(3-pyridylmethylamino)-6-(3-(4-chlorophenyl)propoxy)-3-(2H) pyridazinone; I-methyl-5(5-morpholinoacetyl-2-n-propoxyphenyl)-3-n-propyl-1,6-dihydro-7H-pyrazolo(4,3-d)pyrimidin-7-one; 1-[4-[(1,3-benzodioxol-5-ylmethyl) amino]-6-chloro-2-quinazolinyl]-4-piperidinecarboxylic acid, monosodium salt; Pharmaprojects No. 4516 (Glaxo Wellcome); Pharmaprojects No. 5051 (Bayer); Pharmaprojects No. 5064 (Kyowa Hakko; see WO 96/26940); Pharmaprojects No. 5069 (Schering Plough); GF-196960 (Glaxo Wellcome); E-8010 and E-4010 (Eisai); Bay-38-3045 & 38-9456 (Bayer) and Sch-51866.

Preferably, the PDE5 inhibitor suitable for the present invention is 1-[[3-(6,7-dihydro-1-methyl-7-oxo-3-propyl-1H-pyrazolo [4,3-d]pyrimidin-5-yl)-4-ethoxyphenyl]sulfonyl]-4-methylpiperazine) (sildenafil, sold under the tradename of VIAGRA™, or pharmaceutically acceptable salt thereof, especially sidenafil citrate. A process for its preparation is described in U.S. Pat. No. 6,207,829.

Another preferred PDE5 inhibitor suitable for the present invention is 2-[2-ethoxy-5-(4-ethyl-piperazine-1-sulfonyl)-phenyl]-5-methyl-7-propyl-3H-imidazo[5,1-f][1,2,4]triazin-4-one (vardenafil sold under the tradename of LEVITRA™) (see e.g. U.S. Pat. No. 6,362,178).

Still another preferred PDE5 inhibitor suitable for the present invention is Tadalafil (Pyrazino [1′,2′:1,6]pyrido[3,4-b]indole-1,4-dione,6-(1,3-benzodioxol-5-yl)-2,3,6,7,12,12a-hexahydro-2-methyl-,(6R, 12aR)-6R-trans)-6-(1,3-benzodioxol-5-yl)-2,3,6,7,12,12a-hexahydro-2-methyl-pyrazino[1′,2′:1,6]pyrido[3,4-b]indole-1,4-dione, sold under the tradename CIALIS™) (U.S. Pat. Nos. 5,859,006, 6,140,329).

Other preferred PDE5 inhibitors suitable for the present invention include zaprinast, FR226807, T-1032, KF31327, UK369003, TA1790, DA8159 (Rotella D P. Phosphodiesterase 5 inhibitors: current status and potential applications. Nature Reviews. Drug Discovery. 1(9):674-82, 2002.), and UK122764 (Turko et al., 1999, Inhibition of cyclic CGP-binding cyclic GMP specific phosphodiesterase (type 5) by sildenafil and related compounds. Molecular Pharmacology 56: 124-130).

Other PDE5 inhibitors are discussed in Rotella et al., N-3-substituted imidazoquinazolinones: potent and selective PDE5 inhibitors as potential agents for treatment of erectile dysfunction. Journal of Medicinal Chemistry. 43(7):1257-63, 2000. Rotella et al., Optimization of substituted N-3-benzylimidazoquinazolinone sulfonamides as potent and selective PDE5 inhibitors. Journal of Medicinal Chemistry. 43(26):5037-43, 2000. Kim et al., Synthesis and Phosphodiesterase 5 Inhibitory Activity of New 5-Phenyl-1,6-dihydro-7H-pyrazolo[4,3-d]pyrimidin-7-one derivatives Containing an N-Acylamido Group on a Phenyl Ring. Bioorganic & Medicinal Chemistry 9 1895-1899, 2001.

The suitability of any particular cGMP PDE5 inhibitor can be readily determined by evaluation of its potency and selectivity using methods described in the scientific literature or known to those skilled in the art followed by evaluation of its toxicity, absorption, metabolism, pharmacokinetics, etc. in accordance with standard pharmaceutical practice.

Because the posterior pituitary lies outside the brain, PDE5 inhibitors suitable for the present invention preferably are substances that are impermeable to the blood-brain barrier. Thus they can reach the posterior pituitary to enhance the release of OT or vasopressin without crossing the blood-brain barrier and causing unwanted side effects due to actions in the brain. The posterior pituitary is separated from the blood by a highly permeable capillary endothelium that allows free entry of large charged molecules which cannot permeate the blood-brain barrier.

In order for a substance to enter the brain and spinal cord and produce effects on the central nervous system, it must cross the blood-brain barrier. This generally requires some solubility of the substance in lipids. Conversely, a lipid-insoluble drug will not cross the blood-brain barrier, and will not produce effects on the central nervous system. For example, a compound that acts on the nervous system may be altered to produce a selective peripheral effect by quaternization of the drug, which decreases its lipid solubility and makes it virtually unavailable for transfer to the central nervous system. See e.g. Rowland, M. In: Clinical Pharmacology Basic Principles in Therapeutics (Eds. K. L. Melmon and H. F. Morrelli) Macmillin Co., New York (1972).

Preferably, the cGMP PDE5 inhibitors have an IC50 at less than 100 nanomolar, more preferably, at less than 50 nanomolar, more preferably still at less than 10 nanomolar. IC50 values for the cGMP PDE5 inhibitors may be determined by well-established assays known to those skilled in the art.

Specific methods by which the PDE5 inhibitors, their pharmaceutically acceptable salts and pharmaceutically acceptable solvates, when used in accordance with the invention, may be administered for human clinical or veterinary use, including oral administration by capsule, bolus, tablet or drench, topical administration as an ointment, pour-on, dip, spray, mousse, shampoo, collar or powder formulation, or, alternatively, they can be administered by injection (e.g. subcutaneously, intramuscularly or intravenously), or as an implant. Such formulations may be prepared in a conventional manner in accordance with standard practices well-known to those skilled in the art.

Alternatively, in veterinary use, the PDE5 inhibitors, their pharmaceutically acceptable salts, and pharmaceutically acceptable solvates, when used in accordance with the invention, may be administered with an animal feedstuff and for this purpose a concentrated feed additive or premix may be prepared for mixing with the normal animal feed.

The PDE5 inhibitors, their pharmaceutically acceptable salts, and pharmaceutically acceptable solvates, when used in accordance with the invention, can be administered orally, buccally or sublingually in the form of tablets, capsules (including soft gel capsules), ovules, elixirs, solutions or suspensions, which may contain flavoring or coloring agents, for immediate-, delayed-, modified-, or controlled-release such as sustained-, dual-, or pulsatile delivery applications. The PDE5 inhibitors, their pharmaceutically acceptable salts, and pharmaceutically acceptable solvates, when used in accordance with the invention, may also be administered via fast dispersing or fast dissolving dosage forms or in the form of a dispersion. Suitable pharmaceutical formulations of the PDE5 inhibitors, their pharmaceutically acceptable salts, and pharmaceutically acceptable solvates, when used in accordance with the invention, may be in coated or uncoated form as desired.

Such tablets may contain excipients such as microcrystalline cellulose, lactose, sodium citrate, calcium carbonate, dibasic calcium phosphate, glycine and starch (preferably corn, potato or tapioca starch), disintegrants such as sodium starch glycollate, croscarmellose sodium and certain complex silicates, and granulation binders such as polyvinylpyrrolidone, hydroxypropylmethyl cellulose (HPMC), hydroxypropylcellulose (HPC), sucrose, gelatin and acacia. Additionally, lubricating agents such as magnesium stearate, stearic acid, glyceryl behenate and talc may be included. Physiologically acceptable carriers, excipients, or stabilizers are known to those skilled in the art (see Remington's Pharmaceutical Sciences, 17th edition, (Ed.) A. Osol, Mack Publishing Company, Easton, Pa., 1985). Acceptable carriers, excipients or stabilizers are nontoxic to recipients at the dosages and concentrations employed, and include buffers such as phosphate, citrate, and other organic acids; hydrophobic oils derived from natural or synthetic sources; antioxidants including ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as Tween, Pluronics or polyethylene glycol (PEG).

Preferred excipients in this regard include lactose, starch, cellulose, milk sugar or high molecular weight polyethylene glycols. For aqueous suspensions and/or elixirs, the compounds of the invention may be combined with various sweetening or flavoring agents, coloring matter or dyes, with emulsifying and/or suspending agents and with diluents such as water, ethanol, propylene glycol and glycerin, and combinations thereof.

Modified release and pulsatile release dosage forms may contain excipients such as those detailed for immediate release dosage forms together with additional excipients that act as release rate modifiers, these being coated on and/or included in the body of the device. Release rate modifiers include, but are not limited to, hydroxypropylmethyl cellulose, methyl cellulose, sodium carboxymethylcellulose, ethyl cellulose, cellulose acetate, polyethylene oxide, Xanthan gum, Carbomer, ammonio methacrylate copolymer, hydrogenated castor oil, carnauba wax, paraffin wax, cellulose acetate phthalate, hydroxypropylmethyl cellulose phthalate, methacrylic acid copolymer and mixtures thereof. Modified release and pulsatile release dosage forms may contain one or a combination of release rate modifying excipients. Release rate modifying excipients maybe present both within the dosage form i.e. within the matrix, and/or on the dosage form i.e. upon the surface or coating.

Fast dispersing or dissolving dosage formulations (FDDFs) may contain the following ingredients: aspartame, acesulfame potassium, citric acid, croscarmellose sodium, crospovidone, diascorbic acid, ethyl acrylate, ethyl cellulose, gelatin, hydroxypropylmethyl cellulose, magnesium stearate, mannitol, methyl methacrylate, mint flavouring, polyethylene glycol, fumed silica, silicon dioxide, sodium starch glycolate, sodium stearyl fumarate, sorbitol, xylitol. The terms dispersing or dissolving as used herein to describe FDDFs are dependent upon the solubility of the drug substance used i.e. where the drug substance is insoluble a fast dispersing dosage form can be prepared and where the drug substance is soluble a fast dissolving dosage form can be prepared.

The PDE5 inhibitors, their pharmaceutically acceptable salts, and pharmaceutically acceptable solvates, when used in accordance with the invention, can also be administered parenterally, for example, intravenously, intra-arterially; intraperitoneally, intrathecally, intraventricularly, intraurethrally, intravaginally, intrasternally, intracranially, intramuscularly or subcutaneously, or they may be administered by infusion or needleless injection techniques. For such parenteral administration they are best used in the form of a sterile aqueous solution that may contain other substances, for example, enough salts or glucose to make the solution isotonic with blood. The aqueous solutions should be suitably buffered (preferably to a pH of from 3 to 9), if necessary. The preparation of suitable parenteral formulations under sterile conditions is readily accomplished by standard pharmaceutical techniques well known to those skilled in the art.

The dosage ranges for the administration of pharmaceutical composition of the invention are those large enough to produce the desired effect. The dosage should not be so large as to cause adverse side effects, such as unwanted cross-reactions, anaphylactic reactions, and the like. Generally, the dosage will vary with the age, condition, sex and extent of contion of the patient and can be determined by one of skill in the art. The dosage can be adjusted by the individual physician in the event of any complication.

### EXAMPLES

Materials and Methods

Posterior pituitary. Experiments were performed on posterior pituitary glands removed from rats of either sex, ranging in age from 1-3 months. Animals were rendered unconscious by inhalation of CO₂, and killed by decapitation. Glands were removed rapidly to maintain tissue health, and transferred to physiological saline (in mM: 125 NaCl, 4 KCl, 26 NaHCO₃, 1.25 NaH₂PO₄, 2 CaCl₂, 1 MgCl₂, 10 glucose) bubbled with 95% O₂/5% CO₂ (Jackson et al., 1991, Action potential broadening and frequency-dependent facilitation of calcium signals in pituitary nerve terminals. Proc. Roy. Soc. Lond. 88:380-384). For oxytocin release experiments the pituitary was kept intact. For electrophysiological recordings, slices of posterior pituitary were cut at 70 μm with a vibratome and maintained in physiological saline.

Electrophysiology. Recordings of K⁺ current were made with patch pipettes filled with (in mM) 130 KCl, 10 NaCl, 10 HEPES, 4 Mg-ATP, 0.3 GTP, 5 EGTA, pH 7.3. Individual nerve terminals were identified in an upright DIC microscope, Jackson, 1993. Passive current flow and morphology in the terminal arborizations of the posterior pituitary. J. Neurophysiol. 69, 692-702, and voltage or current clamped with an EPC-7 patch clamp amplifier. K⁺ currents were evoked by 200 ms voltage steps from −80 mV to +50 mV. The slowly inactivating component of K⁺ current represents the activity of BK channels, as verified by comparing with single channel records in cell-attached patches (Bielefeldt, et al., 1992, Three potassium channels in rat posterior pituitary nerve endings. J. Physiol. 458:41-67). Because the A-current in pituitary nerve terminals inactivates almost completely with a time constant of ˜22 msec at 50 mV, the current at the end of pulses >100 msec isolates current through BK channels reasonably well. Nerve terminals were filled with caged cGMP (P-1-(2-nitrophenyl)ethyl ester, 0.5 mM, Calbiochem) by addition to the patch pipette filling solution (Klyachko et al., 2001, cGMP-mediated facilitation in nerve terminals by enhancement of the spike after-hyperpolarization. Neuron 31:1015-1025). This substance was allowed to diffuse into the nerve terminal for 3-4 minutes while making control recordings at regular intervals. Photolysis experiments were performed with illumination from a flash lamp (Rapp Optoelectrik, Hamburg).

Oxytocin release. Whole pituitary glands with attached axon bundle were placed in a chamber perfused with oxygenated physiological saline. Perfusion was stopped at ˜15 min intervals during measurements, when the physiological saline was substituted with a solution of a similar composition, but with the addition of 20 mM HEPES and NaHCO₃, and NaH₂PO₄ reduced to zero. The pH was adjusted to 7.3 with NaOH.

Basal release was measured after a 5 min waiting period (immediately before the stimulus application). The axon bundle was stimulated via a large-diameter, monopolar extracellular glass electrode. Oxytocin release was evoked by a series of 5 trains of 0.2 msec, 0.2 mA current pulses with a frequency of 25 Hz and a duration of 10 seconds. Trains were applied at 1 min intervals. Evoked release was measured 5 minutes after the end of stimulation by gently mixing the bath with a micropipette and collecting 10-20 μL samples in the vicinity of the tissue. Three separate samples were collected for each measurement (including basal release control). Perfusion of the oxygenated physiological saline was restored immediately after sample collection and sildenafil was added to the bath solution for 30-60 min. Recordings of the sildenafil effect on basal and evoked release were then repeated as described above. To control for possible rundown of release, measurements of sildenafil effects were in some cases substituted by the second control measurements.

Oxytocin was measured in collected fluid using the Correlate-EIA enzyme immunoassay kit (Assay Designs, Ann Arbor, Mich.). Oxytocin concentration was inversely proportional to the yellow color generated by the immunoassay and was scanned into the computer using an automated micro plate reader at 405 nm. Actual oxytocin concentration was determined with a standard curve measured for each sample plate.

Results

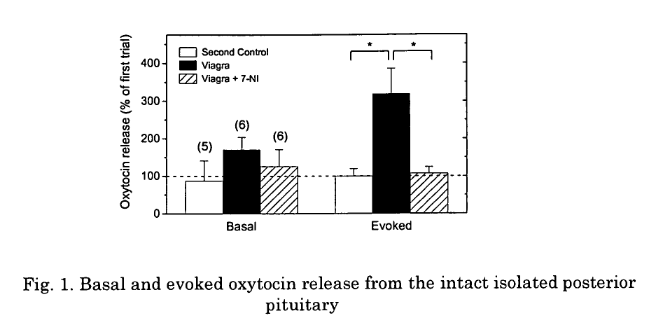

Oxytocin release. FIG. 1 displays measurements of oxytocin release. In the control, no drug was added between the first and second stimulus train, and neither basal nor evoked release was significantly different between the first and second stimulus train. Sildenafil (10 μM) produced an insignificant increase in basal release, which was blocked by 7-NI (7-nitorindazol, 100 μM), an inhibitor of nitric oxide synthase. The same concentration of sildenafil produced a large and significant increase in evoked oxytocin release, which was blocked by 7-NI. These experiments demonstrate that PDE 5 limits the amount of release evoked by electrical stimulation, and blocking this enzyme with sildenafil can enhance evoked oxytocin release.

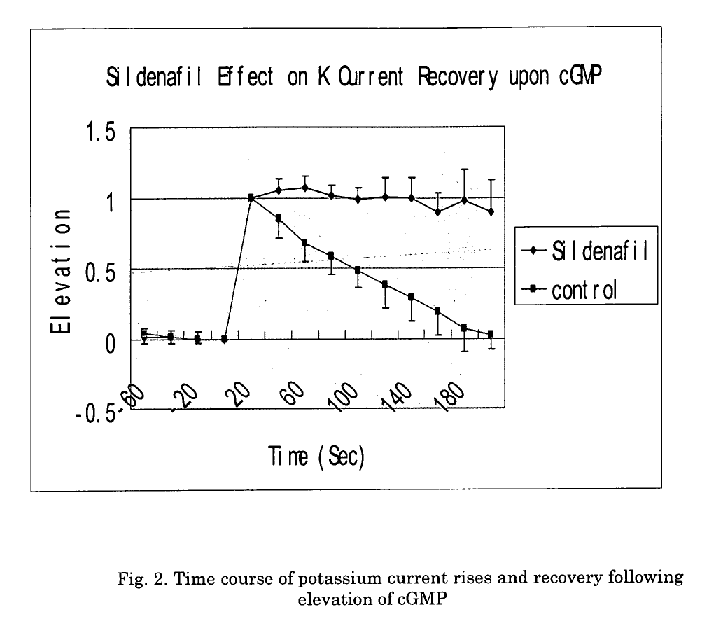

Potassium channel modulation. Patch clamp recordings of potassium current in posterior pituitary slices were made at 20 second intervals. cGMP was introduced by photolysis of caged cGMP. The component of potassium current corresponding to BK channels (seen at the end of a 200 msec voltage step from −80 to 50 mV) was determined for each pulse. The current evoked by the pulse at zero time was subtracted from current at other times and changes were normalized to the maximum seen immediately after cGMP release (FIG. 2). When slices were bathed in 15 μM sildenafil, the increase in potassium current induced by cGMP failed to recover. In control experiments with no added drug the potassium current recovered in about 3 minutes.

FIG. 2 shows that the cGMP-induced increase in potassium current through the pituitary nerve terminal membrane is terminated by the action of PDE5. Sildenafil inhibits this enzyme, greatly slowing the recovery potassium current. The work of Klyachko et al (2001) showed that the cGMP-induced increase in potassium current is uniquely tailored to enhance the electrical excitability of the nerve terminal. Ordinarily, action potentials in the pituitary nerve terminal start to fail as a train of high frequency stimulation pulses is applied. After 5 or 10 seconds, only ˜30% of the stimulus pulses evoke action potentials in control experiments. However, after elevating cGMP the success rate increases to ˜60% (see FIG. 7d of Klyachko et al., 2001). The increase in excitability allows action potentials propagating along the axon bundle to penetrate the posterior pituitary more effectively. The enhancement by sildenafil of the effect of cGMP on potassium current (FIG. 2) thus explains the enhancement of oxytocin release by sildenafil (FIG. 1).

The foregoing description and examples have been set forth merely to illustrate the invention and are not intended to be limiting. Since modifications of the disclosed embodiments incorporating the spirit and substance of the invention may occur to persons skilled in the art, the invention should be construed broadly to include all variations falling within the scope of the appended claims and equivalents thereof. All references cited hereinabove and/or listed below are hereby expressly incorporated by reference.

We claim:

1. A method for increasing the release of oxytocin or vasopressin from the posterior pituitary of a mammal in need thereof, the method comprising administering to the mammal an effective amount of a cyclic guanosine 3′,5′-monophosphate phosphodiesterase type five (cGMP PDE5) inhibitor.

2. The method according to claim 1, wherein the mammal is a pregnant female mammal and wherein labor or fetal expulsion is induced, enhanced or augmented.

3. The method according to claim 2 wherein the female mammal is near-term, full-term or over-term pregnant and wherein labor is induced or enhanced.

4. The method according to claim 3 wherein labor is induced.

5. The method according to claim 3 wherein labor is enhanced.

6. The method according to claim 2 wherein the female mammal is a pig or cow.

7. The method according to claim 2, wherein the mammal is a human.

8. The method according to claim 1, wherein the mammal is a prenatal, neonatal or postnatal female mammal and wherein milk let-down is induced, enhanced or augmented.

9. The method according to claim 8 wherein the female mammal is a pig or cow.

10. The method according to claim 8, wherein the mammal is a human.

11. The method according to claim 1, wherein the PDE5 inhibitor is selected from the group consisting of: pyrazolo (4,3-d)pyrimidin-7-ones; isomeric pyrazolo (3,4-d)pyrimidin-4-ones; quinazolin-4-ones; pyrido (3,2-d)pyrimidin-4-ones; purin-6-ones; and pyrazolo (4,3-d)pyrimidin-4-ones, in the form of a racemate, pure stereoisomer, or in the form of a mixture of stereoisomers in any mixing ratio.

12. The method according to claim 11, wherein the stereoisomer is an enantiomer or a diastereomer.

13. The method according to claim 1 wherein the PDE V inhibitor is 3-ethyl-5-(5-(4-ethylpiperazin-1-ylsulphonyl)-2-n-propoxyphenyl)-2-(pyridin-2-yl)methyl-2,6-dihydro-7H-pyrazolo(4,3-d)pyrimidin-7-one or sildenafil.

14. The method according to claim 1 wherein the PDE V inhibitor is vardenafil (2-[2-ethoxy-5-(4-ethyl-piperazine-1-sulfonyl)-phenyl]-5-methyl-7-propyl-3H-imidazo[5,1-f][1,2,4]triazin-4-one).

15. The method according to claim 1 wherein the PDE V inhibitor is Tadalafil (Pyrazino[1′,2′:1,6]pyrido[3,4-b]indole-1,4-dione,6-(1,3-benzodioxol-5-yl)-2,3,6,7,12,12a-hexahydro-2-methyl-,(6R, 12aR)-6R-trans)-6-(1,3-benzodioxol-5-yl)-2,3,6,7,12,12a-hexahydro-2-methyl-pyrazino[1′,2′:1,6]pyrido[3,4-b]indole-1,4-dione).

16. The method according to claim 1 wherein the PDE V inhibitor is selected from the group consisting of zaprinast, UK122764, FR226807, T-1032, KF31327, UK369003, TA1790, and DA8159.

17. A method for increasing the release of oxytocin or vasopressin from the posterior pituitary of a mammal in need thereof, the method comprising administering to the mammal an effective amount of sildenafil.

18. The method according to claim 17, wherein the mammal is a pregnant female mammal and wherein labor or fetal expulsion is induced, enhanced or augmented, or wherein the mammal is a prenatal, neonatal or postnatal female mammal and wherein milk let-down is induced, enhanced or augmented.

19. The method according to claim 18, wherein the female mammal is a human or a dairy animal.

20. A pharmaceutical composition for a treatment method for increasing the release of oxytocin or vasopressin from the posterior pituitary of a mammal in need thereof, the pharmaceutical composition comprising an effective amount of a cyclic guanosine 3′,5′-monophosphate phosphodiesterase type five (cGMP PDE5) inhibitor, and a pharmaceutically acceptable excipient.
